(19) United States
(12) Patent Application Publication
(10) Pub. No.: US 2021/0153727 A1
(43) Pub. Date: May 27, 2021

(54) MEDICAL MANIPULATOR SYSTEM AND ACCESS DEVICE

(71) Applicant: OLYMPUS CORPORATION, Tokyo (JP)

(72) Inventor: Mitsuaki HASEGAWA, Tokyo (JP)

(73) Assignee: OLYMPUS CORPORATION, Tokyo (JP)

(21) Appl. No.: 17/167,294

(22) Filed: Feb. 4, 2021

Related U.S. Application Data
(63) Continuation of application No. PCT/JP2018/031340, filed on Aug. 24, 2018.

(51) Int. Cl. A61B 1/01; A61B 1/00; A61B 1/005

(52) U.S. Cl. CPC A61B 1/01; A61B 1/00087; A61B 1/00006; A61B 1/00154; A61B 1/0051

(57) ABSTRACT

A medical manipulator system is equipped with a treatment tool and an access device that guides the treatment tool. The treatment tool includes a flexible shaft equipped with a treatment unit at a distal end, a rigid shaft connected to a proximal end of the flexible shaft, and an operation unit connected to a proximal end of the rigid shaft and configured to operate the treatment unit. The access device includes a flexible tube into which the flexible shaft is to be inserted, and a rigid unit connected to a proximal end of the flexible tube. The rigid unit includes an insertion hole into which the rigid shaft is to be inserted. An inner surface of the insertion hole has a projection that projects radially inward. The projection faces an outer circumferential surface of the rigid shaft inserted into the insertion hole.

### CROSS-REFERENCE TO RELATED APPLICATIONS

This is a continuation of International Application PCT/JP2018/031340, with an international filing date of Aug. 24, 2018, which is hereby incorporated by reference herein in its entirety.

### TECHNICAL FIELD

The present invention relates to a medical manipulator system and an access device.

### BACKGROUND ART

There is known a medical manipulator system that includes a treatment tool and an access device, in which the treatment tool includes a flexible shaft having a treatment unit at a distal end, a rigid shaft disposed at a proximal end of the flexible shaft, and an operation unit disposed at a proximal end of the rigid shaft and configured to operate the treatment unit, and the access device includes a flexible tube into which the flexible shaft is to be inserted and a rigid pipe into which the rigid shaft is to be inserted (for example, refer to United States Patent Application No. 2015/0150633).

By moving the rigid shaft within the rigid pipe, the operation unit is guided to advance or retract. Thus, the flexible shaft can be advanced and retracted relative to the flexible pipe without buckling, and the operation force of the operation unit can be efficiently transmitted to the treatment unit projecting from the distal end of the flexible pipe.

### SUMMARY OF INVENTION

An aspect of the present invention provides a medical manipulator system equipped with a treatment tool and an access device that guides the treatment tool. The treatment tool includes a flexible shaft equipped with a treatment unit at a distal end, a rigid shaft connected to a proximal end of the flexible shaft, and an operation unit connected to a proximal end of the rigid shaft and configured to operate the treatment unit. The access device includes a flexible tube having an inner lumen into which the flexible shaft is to be inserted, and a rigid unit connected to a proximal end of the flexible tube. The rigid unit includes an insertion hole into which the rigid shaft is to be inserted and which is connected to the inner lumen. An inner surface of the insertion hole has a projection that is disposed on a proximal end side with respect to the center position in the longitudinal direction and that projects radially inward. The projection faces an outer circumferential surface of the rigid shaft inserted into the insertion hole.

### BRIEF DESCRIPTION OF DRAWINGS

FIG. 1 is an overall structural diagram of a medical manipulator system according to one embodiment of the present invention.

FIG. 2 is a side view illustrating a treatment tool of the medical manipulator system illustrated in FIG. 1.

FIG. 3 is a perspective view illustrating an access device according to an embodiment of the present invention in the medical manipulator system illustrated in FIG. 1.

FIG. 4 is a longitudinal sectional view illustrating the relationship between the access device illustrated in FIG. 3 and the treatment tool illustrated in FIG. 2.

FIG. 5 is a partial longitudinal sectional view illustrating projections in a rigid cylinder unit of the access device illustrated in FIG. 1.

FIG. 6 is a longitudinal sectional view illustrating the state in which the treatment tool is advanced relative to the access device illustrated in FIG. 4.

FIG. 7 is a transversal sectional view taken at a first position illustrated in FIG. 6.

FIG. 8 is a transversal sectional view of a first modification of the access device illustrated in FIG. 3 taken at the first position.

FIG. 9 is a longitudinal sectional view illustrating a second modification of the access device illustrated in FIG. 3.

FIG. 10 is a transversal sectional view taken at a first position illustrated in FIG. 9.

FIG. 11 is a transversal sectional illustrating a third modification of the access device illustrated in FIG. 3 taken at the first position.

FIG. 12 is a partial longitudinal sectional view illustrating a fourth modification of the access device illustrated in FIG. 3.

### DESCRIPTION OF EMBODIMENTS

A medical manipulator system 100 and an access device 1 according to an embodiment of the present invention will now be described with reference to the drawings.

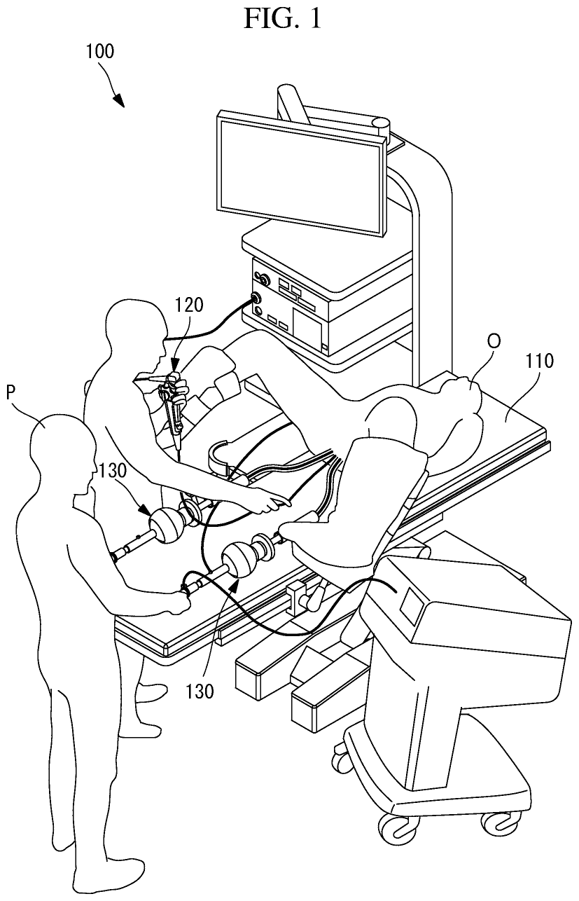

As illustrated in FIG. 1, the medical manipulator system 100 of this embodiment includes: the access device 1 of this embodiment inserted into the body cavity together with an endoscope 120 through the anus of a patient O lying on an operating table 110; and two treatment tools 130 that are guided by the access device 1 and inserted into the body cavity.

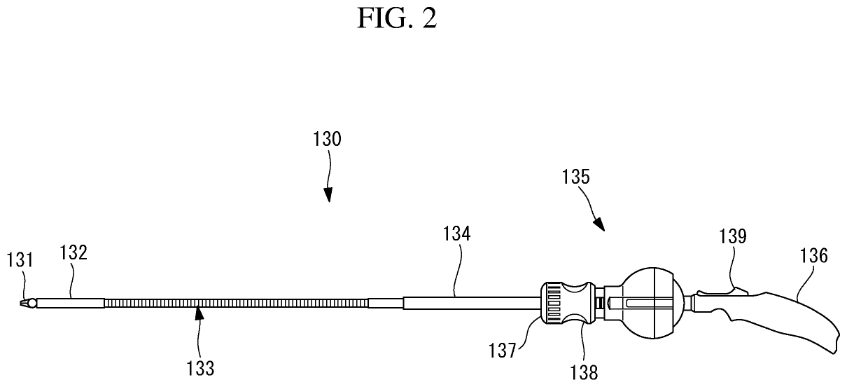

As illustrated in FIG. 2, each of the treatment tools 130 is equipped with, in order from the distal end, a flexible shaft 133 that has a grasping unit (treatment unit) 131 and a bending unit (treatment unit) 132, a rigid shaft 134 connected to a proximal end of the flexible shaft 133, and an operation unit 135 connected to a proximal end of the rigid shaft 134 and configured to operate the grasping unit 131.

The operation unit 135 is equipped with a grip 136 to be held by the operator P and a force conversion mechanism 137 that converts a rotation operation applied to the grip 136 into bending movement of the bending unit 132. The force conversion mechanism 137 and the rigid shaft 134 are rotatably connected to each other about the longitudinal axis of the rigid shaft 134. A lock mechanism 138 that can lock the rotation of the force conversion mechanism 137 and the rigid shaft 134 at a desired position is also provided. In the drawings, reference sign 139 denotes a slider used by the operator P to open and close the grasping unit 131.

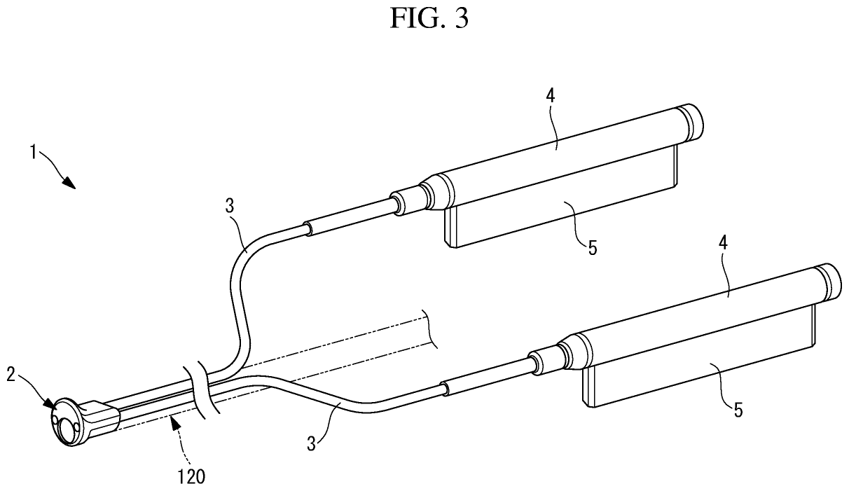

As illustrated in FIG. 3, the access device 1 according to this embodiment is equipped with an endoscope attachment unit 2 to be attached to the distal end of the endoscope 120, two flexible tubes 3 having distal ends connected to the endoscope attachment unit 2, and rigid cylinder units (rigid unit) 4 connected to the proximal ends of the flexible tubes 3. Each of the rigid cylinder units 4 is equipped with a fixing unit 5 used to fix the rigid cylinder unit 4 to the operating table 110.

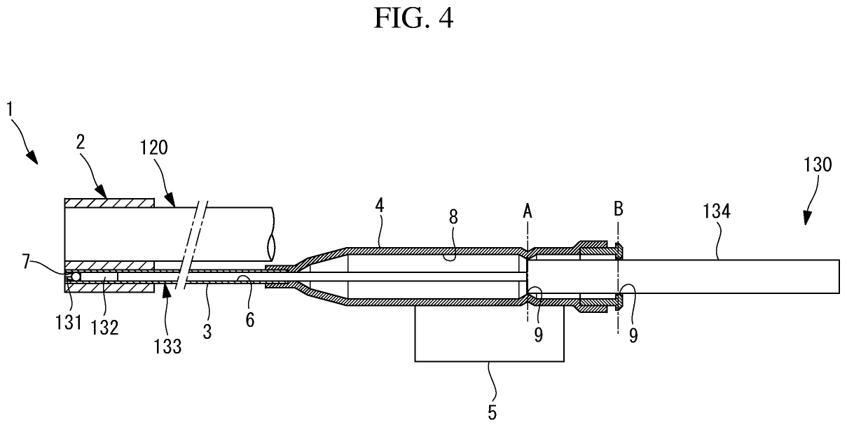

As illustrated in FIG. 4, the flexible tube 3 has an inner lumen 6 into which the flexible shaft 133 of the treatment tool 130 is inserted so as to be advanceable and retractable in the longitudinal axis direction. The flexible tube 3 has a distal end opening 7 in the distal end surface of the endoscope attachment unit 2. The rigid cylinder unit 4 has an insertion hole 8 connected to the inner lumen 6 of the flexible tube 3 so that the rigid shaft 134 of the treatment tool 130 is inserted so as to be advanceable and retractable in the longitudinal axis direction.

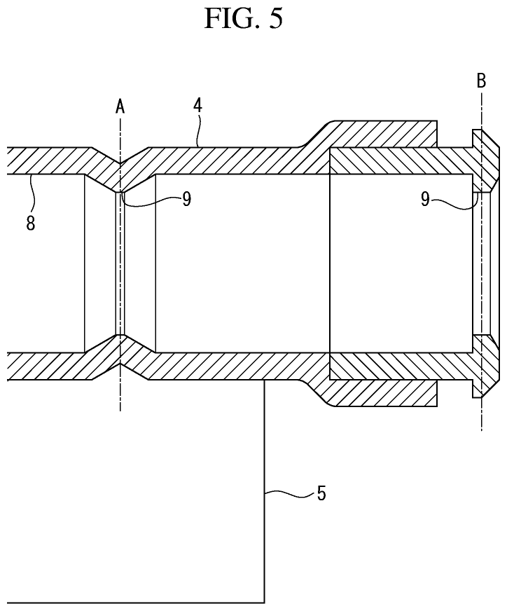

In this embodiment, as illustrated in FIGS. 4 and 5, an inner surface of the rigid cylinder unit 4 has annular projections 9 that each project radially inward around the entire circumference, and these projections 9 are provided at two positions, namely, a most-proximal position (second position) B of the rigid cylinder unit 4 and a position (first position) A spaced from the position B and located on the distal end side relative to the position B in the axis direction. The projections 9 have an inner diameter slightly larger than the outer diameter of the rigid shaft 134 of the treatment tool 130.

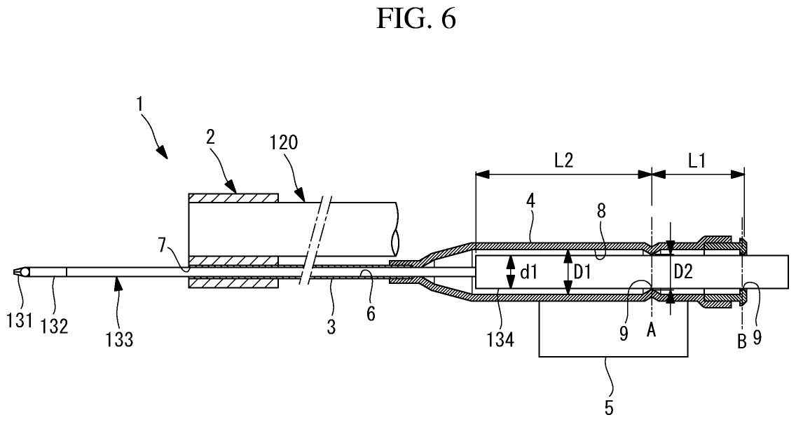

Specifically, the inner diameter of the rigid cylinder unit 4, the inner diameters of the two projections 9, the distance between the two projections 9 in the axis direction, and the outer diameter of the rigid shaft 134 are set to such dimensions that the distal end of the rigid shaft 134 is guaranteed to not contact the inner surface of the rigid cylinder unit 4 as long as the rigid shaft 134 is inserted into the rigid cylinder unit 4 until the rigid shaft 134 passes the two projections 9. For example, as illustrated in FIG. 6, when the inner diameter D1 of the rigid cylinder unit 4, the inner diameter D2 of the two projections 9, the length L1 between the second position B and the first position A, which is the distance between the two projections 9 in the axis direction, and the outer diameter d1 of the rigid shaft 134 take particular values and when the maximum insertion amount of the rigid shaft 134 from the projection 9 at the first position is L2, the following conditional formula (1) is satisfied:

(D1−D2)/(2×L2)>(D2−d1)/L1 (1)

In this embodiment, as illustrated in FIG. 4, the projection 9 at the first position A is present at a position of the distal end of the rigid shaft 134 when the distal end of the grasping unit 131 at the distal end of the treatment tool 130 is in alignment with the distal end opening 7 in the endoscope attachment unit 2. In this manner, when the treatment tool 130 is advanced and the grasping unit 131 becomes exposed forward from the distal end opening 7, the rigid shaft 134 always assumes a state in which the projections 9 at the two positions face the outer circumferential surface of the rigid shaft 134. In other words, the movement of the outer circumferential surface of the rigid shaft 134 is restricted in the radial direction by the two projections 9 at the two positions irrespective of whether the treatment tool 130 is in a state illustrated in FIG. 4, in a state illustrated in FIG. 6 in which the treatment tool 130 is advanced farthest, or in any state between the state illustrated in FIG. 4 and the state illustrated in FIG. 6.

The operation of the medical manipulator system 100 and the access device 1 of this embodiment having such features will now be described.

In order to treat an affected site inside the body of the patient O by using the medical manipulator system 100 of this embodiment, a distal end of the endoscope 120 is attached to the endoscope attachment unit 2 of the access device 1, and two treatment tools 130 are respectively inserted into the proximal end openings of the insertion holes 8 of the two rigid cylinder units 4. Each of the treatment tools 130 is inserted until the flexible shaft 133 is inside the flexible tube 3.

In this state, the access device 1 and the distal end of the endoscope 120 are inserted from the anus into the large intestine of the patient O lying on the operating table 110. While observing the state of the large intestine through the endoscopic image, the endoscope 120 and the access device 1 are advanced inside the large intestine to a position where the affected site is within the field of view of the endoscope 120. In this state, the fixing units 5 of the rigid cylinder units 4 of the access device 1 are fixed to the operating table 110 so that the operation units 135 of the two treatment tools 130 are at relative positions with which one operator P can easily manipulate the operation units 135 with both of his/her hands. Here, “fixed to the operating table 110” encompasses the instance where the fixing units 5 are directly fixed to the operating table 110 and the instance where the fixing units are fixed to arms that can be placed at positions fixed to the operating table 110.

Next, each of the treatment tools 130 is advanced within the access device 1, and the rigid shaft 134 is inserted into the insertion hole 8 of the rigid cylinder unit 4 from the proximal end side. The angle between the rigid shaft 134 and the longitudinal axis of the rigid cylinder unit 4 can be relatively freely determined at the time point where the rigid shaft 134 is inserted over the projection 9 at the second position B. In contrast, as the distal end of the rigid shaft 134 approaches the first position A, the inner circumferential surfaces of the two projections 9 at the positions A and B spaced from each other in the longitudinal axis direction come close to the outer circumferential surface of the rigid shaft 134; thus, the angle between the rigid shaft 134 and the longitudinal axis of the rigid cylinder unit 4 is limited to an extremely narrow angle range depending on the clearance between the inner circumferential surfaces of the projections 9 and the outer circumferential surface of the rigid shaft 134.

When the rigid shaft 134 is further advanced from this position A, the grasping unit 131 projects from the distal end opening 7 of the access device 1 and enters the field of view of the endoscope 120; thus, the operator P can treat the affected site by manipulating the operation unit 135. The operator P adjusts the angular position of the grasping unit 131 about the longitudinal axis while looking at the endoscopic image, and locks the position through the lock mechanism 138. Next, the operator P holds the grip 136 and rotates the grip 136 about an axis intersecting the longitudinal axis of the rigid shaft 134 so that this rotation amount is converted into the bending angle of the bending unit 132 at the distal end of the flexible shaft 133 through the force conversion mechanism 137, thereby causing the bending unit 132 to bend. The operator P can open and close the grasping unit 131 by manipulating the slider 139 installed in the grip 136 and treat the tissue at the affected site.

In this case, since the movement of the rigid shaft 134 in the radial direction is always restricted by the two projections 9 in the state in which the grasping unit 131 projects from the distal end opening 7 of the endoscope attachment unit 2, the operation of advancing and retracting the treatment tool 130 in the longitudinal direction relative to the access device 1 can be stabilized despite the direction and the magnitude of the force applied to the grip 136. That is, in a state in which the grasping unit 131 projects from the distal end opening 7 of the endoscope attachment unit 2, it is always the case that even when the rigid shaft 134 is slanted to a maximum point with respect to the longitudinal axis of the rigid cylinder unit 4 inside the rigid cylinder unit 4 of the access device 1, the distal end of the rigid shaft 134 does not contact the inner surface of the rigid cylinder unit 4; thus, a rapid increase in friction between the treatment tool 130 and the access device 1 can be prevented.

As a result, in a state in which the operator P treats the affected site with the treatment tools 130, the force needed to advance or retract the treatment tools 130 does not fluctuate; thus, there is an advantage in that the operational feeling of the operator P remains unchanged. In other words, when the frictional resistance rapidly increases as the treatment tools 130 advances, the operator P intuitively and falsely recognizes that the grasping unit 131 has come into contact with the tissue and is thus forced to perform the subsequent operation with greater caution. In contrast, this embodiment avoids such an inconvenience and provides an advantage in that the operator P can perform treatment intuitively.

Specifically, depending on the gravitational force applied to the treatment tool 130 or the direction of the force applied to the grip 136, a force that causes the rigid shaft 134 to lean in the radial direction or slant with respect to the longitudinal axis acts inside the rigid cylinder unit 4; however, according to this embodiment, the relationship between the rigid cylinder unit 4 and the rigid shaft 134 can be retained constant under such conditions, and there is an advantage in that stable operation is possible.

Another advantage is that, because the projections 9 are disposed on the proximal end side with respect to the center of the rigid cylinder unit 4 in the longitudinal axis direction, the orientation of the rigid shaft 134 can be stabilized from the initial stage of inserting the rigid shaft 134 into the rigid cylinder unit 4.

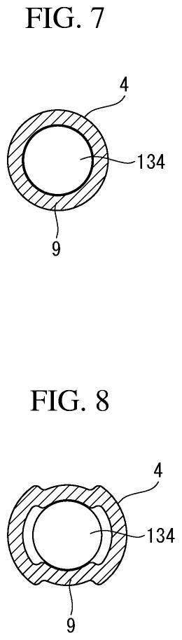

As illustrated in FIG. 7, one of the projections 9 described so far in this embodiment is a projection that projects from a rigid cylinder member 4 having a substantially perfectly circular cross-sectional shape and is formed at the first position A around the entire circumference so that the inner diameter of the rigid cylinder member 4 is smaller at the first position A; alternatively, as illustrated in FIG. 8, the projection 9 may have a partially projecting form in which a portion of the rigid cylinder unit 4 in the circumferential direction projects inward at the first position A and another portion of the rigid cylinder unit 4 opposing the aforementioned portion with the rigid shaft 134 therebetween also projects inward at the first position A. Instead of the rigid cylinder unit 4 having inwardly projecting portions at the two positions opposing each other with the rigid shaft 134 therebetween at the first position A of the rigid cylinder unit 4, the rigid cylinder unit 4 may have inwardly projecting portions at three or more positions spaced from one another in the circumferential direction. This may apply not only to the projection 9 at the first position A but also to the projection 9 at the second position B.

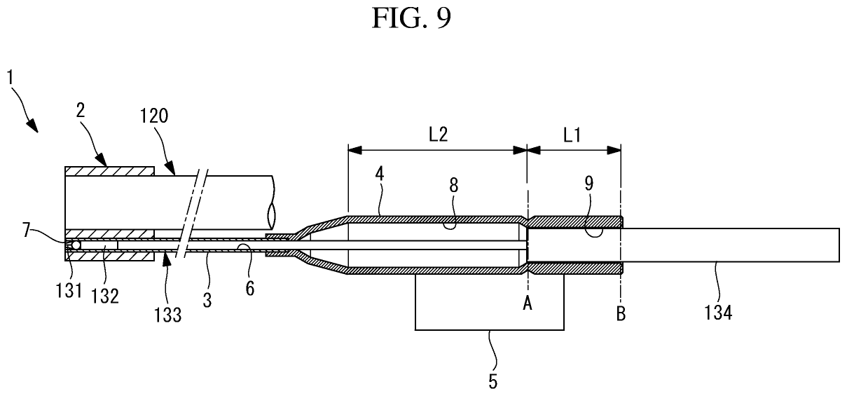

In this embodiment, the projections 9 are formed on the inner surface of the rigid cylinder unit 4 at two positions A and B spaced from each other in the longitudinal axis direction; however, this embodiment is not limited to this feature as long as the projections 9 oppose the outer circumferential surface of the rigid shaft 134 at at least two positions spaced from each other in a direction along the axis of the insertion hole 8. For example, the projections 9 may be formed at three or more positions, or, as illustrated in FIG. 9, one projection 9 may be formed over a particular length range in the longitudinal axis direction.

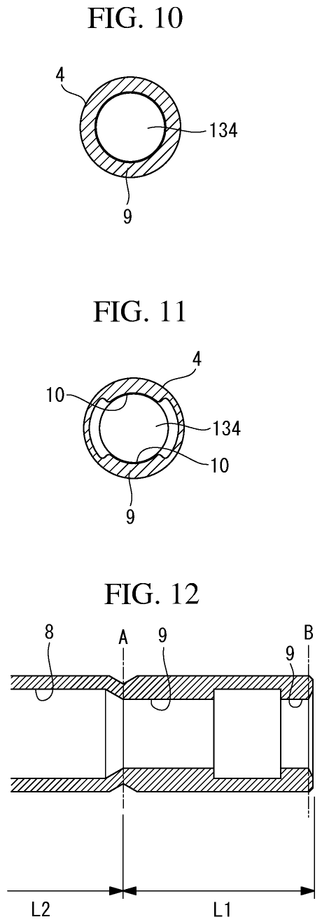

As illustrated in FIG. 10, the one projection 9 formed over a particular length range in the longitudinal axis direction has the feature that the entire inner diameter of the rigid cylinder unit 4 is decreased; alternatively, as illustrated in FIG. 11, the projection 9 may include a step portion 10 that projects inward over a particular length range in the longitudinal axis direction in one part in the circumferential direction of the rigid cylinder unit 4, and another step portion 10 opposing the aforementioned step portion 10 with the rigid shaft 134 therebetween. The step portions 10 may be provided at three or more positions at particular intervals in the circumferential direction.

When more than one projections 9 are formed, as illustrated in FIG. 12, the projections 9 may each be formed over a particular length range in the longitudinal axis direction. However, when the length of the inner circumferential surface of the projection 9 in the longitudinal axis direction is large, the friction between the outer circumferential surface of the rigid shaft 134 and the projection 9 increases. Thus, the length is preferably small.

The first position described above as an example is a position where the distal end of the rigid shaft 134 lies when the grasping unit 131 is in alignment with the distal end of the endoscope attachment unit 2; however, the first position may be set closer to the proximal end than this position. This is because when the grasping unit 131 is placed inside the flexible tube 3 and the rigid shaft 134 is caused to pass over the two projections 9, the orientation of the rigid shaft 134 is stably supported when the grasping unit 131 projects from the distal end opening 7.

Although an annular projection provided on the inner surface of the rigid cylinder unit 4 around the entire circumference is described above as an example of the projection 9, the projection 9 may be provided only in some part of the inner surface of the rigid cylinder unit 4 in the circumferential direction if the direction in which the rigid shaft 134 leans inside the rigid cylinder unit 4 is limited.

As a result, the following aspect is read from the above described embodiment of the present invention.

An aspect of the present invention provides a medical manipulator system that includes a treatment tool and an access device configured to guide the treatment tool. The treatment tool includes a flexible shaft equipped with a treatment unit at a distal end thereof, a rigid shaft connected to a proximal end of the flexible shaft, and an operation unit connected to a proximal end of the rigid shaft and configured to operate the treatment unit. The access device includes a flexible tube having an inner lumen into which the flexible shaft is to be inserted and a rigid unit connected to a proximal end of the flexible tube. The rigid unit includes an insertion hole into which the rigid shaft is to be inserted and which is connected to the inner lumen. Projections each projecting radially inward and having a particular length in a longitudinal axis direction are provided on an inner surface of the insertion hole, and the projections are respectively provided at at least two positions spaced from each other in a direction along an axis of the insertion hole.

According to this aspect, when the flexible shaft of the treatment tool is inserted into the inner lumen of the flexible tube of the access device and the rigid shaft of the treatment tool is inserted into the insertion hole of the rigid unit of the access device, the movement of the rigid shaft in the longitudinal direction is guided by the insertion hole; thus, the flexible shaft can be moved in the longitudinal direction without buckling, and the treatment tool can be advanced and retracted to cause the treatment tool to emerge from and withdraw into the distal end opening of the

In this case, a particular clearance is present between the insertion hole and the rigid shaft. When the rigid shaft inserted into the insertion hole approaches the projections formed on the inner surface of the insertion hole, the projections are positioned to be close to the outer circumferential surface of the rigid shaft. Thus, during the initial stage of inserting the rigid shaft into the insertion hole, the movement of the distal end of the rigid shaft in the radial direction is limited within the insertion hole. As a result, compared to when there are no projections, the inclination angle of the rigid shaft with respect to the axis of the insertion hole is suppressed to a low angle, and the insertion resistance generated by interference of the distal end of the rigid shaft against the inner surface of the insertion hole can be reduced. As a result, the change in insertion resistance corresponding to the insertion amount of the rigid shaft into the rigid pipe is decreased, and constant operational feeling can be obtained irrespective of the insertion amount.

In the aspect described above, the flexible tube may have a distal end opening from which the treatment unit projects; and when the rigid shaft and the rigid unit are in such a positional relationship that allows the treatment unit to project from the distal end opening, the projections at the at least two positions spaced from each other in the direction along the axis of the insertion hole may always face an outer circumferential surface of the rigid shaft.

According to this feature, the movement of the rigid shaft in the radial direction is restricted by the projections at the two positions spaced from each other in the direction along the axis of the insertion hole. When the treatment unit is positioned to project from the distal end opening, in other words, when the operator performs the treatment by using the treatment unit, the inclination angle of the rigid shaft with respect to the insertion hole can be always maintained at a constant angle, and thus constant operational feeling can be obtained irrespective of the insertion amount of the rigid shaft into the rigid pipe.

In the aspect described above, the projections may be disposed at a first position and a second position spaced from the first position and located on a proximal end side relative to the first position, the first position being a position of a distal end of the rigid shaft in the longitudinal direction when a distal end of the treatment unit is in alignment with the distal end opening, or a position on the proximal end side relative to the aforementioned position.

According to this feature, when the distal end of the treatment unit is withdrawn from the distal end opening and is located on the proximal end side relative to the distal end opening, only the projection at the second position faces the outer circumferential surface of the rigid shaft; however, when the distal end of the treatment unit is in alignment with the distal end opening, the projections at the two positions, the first position and the second positions, face the outer circumferential surface of the rigid shaft. As a result, the inclination angle of the rigid shaft in the insertion hole is restricted to a particular angle. In addition, when the rigid shaft is further advanced toward the distal end side and the treatment unit is caused to project from the treatment unit, the same state in which the projections at the two positions, the first position and the second position, face the outer peripheral surface of the rigid shaft can be maintained.

In the aspect described above, conditional formula (1) below may be satisfied:

(D1−D2)/(2×L2)>(D2−d1)/L1 (1)

where
D1 represents an inner diameter of the rigid unit;
D2 represents an inner diameter of the two projections;
L1 represents a length from the second position to the first position;
L2 represents a maximum insertion amount of the rigid shaft from the first position; and
d1 represents an outer diameter of the rigid shaft.

In the aspect described above, the operation unit may be equipped with a grip to be held by an operator, and a force conversion mechanism configured to convert a rotation operation applied to the grip into movement of the treatment tool.

According to this feature, when the operator holds the grip and performs rotation operation, the treatment tool is moved by the force conversion mechanism. In such a case, the projections approach the outer circumferential surface of the rigid shaft and restrict the movement of the distal end of the rigid shaft in the radial direction in the insertion hole; thus, rattling during the rotation operation can be reduced, and the operability can be improved.

In the aspect described above, the force conversion mechanism and the rigid shaft may be rotatably connected to each other about a longitudinal axis of the rigid shaft, and the medical manipulator system may further include a lock mechanism configured to lock rotation of the force conversion mechanism and the rigid shaft.

According to this feature, after the grip rotation direction and the phase of the rigid shaft about the longitudinal axis are adjusted by releasing the lock mechanism, the rotation of the force conversion mechanism and the rigid shaft can be locked by the lock mechanism so that the rotational force about the axis of the rigid shaft applied to the grip can cause the treatment unit at the distal end of the flexible shaft to undergo roll rotation.

Another aspect of the present invention provides an access device configured to guide a treatment tool that includes a flexible shaft equipped with a treatment unit at a distal end thereof, and a rigid shaft connected to a proximal end of the flexible shaft. The access device includes a flexible tube having an inner lumen into which the flexible shaft is to be inserted, and a rigid unit connected to a proximal end of the flexible tube. The rigid unit includes an insertion hole into which the rigid shaft is to be inserted and which is connected to the inner lumen. Projections each projecting radially inward and having a particular length in a longitudinal axis direction are provided on an inner surface of the insertion hole, and the projections are respectively provided at at least two positions spaced from each other in a direction along an axis of the insertion hole.

In the aspect described above, the flexible tube may have a distal end opening from which the treatment unit projects; and when the rigid shaft and the rigid unit are in such a positional relationship that allows the treatment unit to project from the distal end opening, the projections at the at least two positions spaced from each other in the direction along the axis of the insertion hole may always face an outer circumferential surface of the rigid shaft.

Yet another aspect of the present invention provides a medical manipulator system including a treatment tool and an access device configured to guide the treatment tool. The treatment tool includes a flexible shaft equipped with a treatment unit at a distal end thereof, a rigid shaft connected to a proximal end of the flexible shaft, and an operation unit connected to a proximal end of the rigid shaft and configured to operate the treatment unit. The access device includes a flexible tube having an inner lumen into which the flexible shaft is to be inserted and a rigid unit connected to a proximal end of the flexible tube. The rigid unit includes an insertion hole into which the rigid shaft is to be inserted and which is connected to the inner lumen. An inner diameter of the insertion hole on a proximal end side in the longitudinal direction is smaller than that on a distal end side in the longitudinal direction.

In the aspect described above, a projection projecting radially inward and having a particular length in a longitudinal axis direction may be formed on an inner surface of the insertion hole, the projection being disposed on a proximal end side with respect to a center position in the longitudinal direction, and an inner diameter of the insertion hole at a position where the projection is located may be smaller than that on a distal end side in the longitudinal direction.

In the aspect described above, the projection may have a length that spans from a first position to a second position spaced from the first position and located on a proximal end side relative to the first position, the first position being a position of a distal end of the rigid shaft in the longitudinal direction when a distal end of the treatment unit is in alignment with the distal end opening, or a position on the proximal end side relative to the aforementioned position.

In the aspect described above, the projection may include projections disposed at a first position and a second position spaced from the first position and located on a proximal end side relative to the first position, the first position being a position of a distal end of the rigid shaft in the longitudinal direction when a distal end of the treatment unit is in alignment with the distal end opening, or a position on the proximal end side relative to the aforementioned position.

In the aspect described above, the operation unit may be equipped with a grip to be held by an operator and a force conversion mechanism configured to convert a rotation operation applied to the grip into movement of the treatment tool.

In the aspect described above, the force conversion mechanism and the rigid shaft may be rotatably connected to each other about a longitudinal axis of the rigid shaft, and the medical manipulator system may further include a lock mechanism configured to lock rotation of the force conversion mechanism and the rigid shaft.

### REFERENCE SIGNS LIST

1 access device
3 flexible tube
4 rigid cylinder unit (rigid unit)
6 inner lumen
7 distal end opening
8 insertion hole
9 projection
100 medical manipulator system
130 treatment tool
131 grasping unit (treatment unit)
132 bending unit (treatment unit)
133 flexible shaft
134 rigid shaft
135 operation unit
136 grip
137 force conversion mechanism
138 lock mechanism
A first position
B second position
P operator

1. A medical manipulator system comprising:
a treatment tool; and
an access device configured to guide the treatment tool,
wherein the treatment tool includes
a flexible shaft equipped with a treatment unit at a distal end thereof,
a rigid shaft connected to a proximal end of the flexible shaft, and
an operation unit connected to a proximal end of the rigid shaft and configured to operate the treatment unit;
the access device includes
a flexible tube having an inner lumen into which the flexible shaft is to be inserted, and
a rigid unit connected to a proximal end of the flexible tube; and
the rigid unit includes
an insertion hole into which the rigid shaft is to be inserted and which is connected to the inner lumen;
projections each projecting radially inward and having a particular length in a longitudinal axis direction are provided on an inner surface of the insertion hole; and
the projections are respectively provided at at least two positions spaced from each other in a direction along an axis of the insertion hole.

2. The medical manipulator system according to claim 1, wherein:
the flexible tube has a distal end opening from which the treatment unit projects; and
when the rigid shaft and the rigid unit are in such a positional relationship that allows the treatment unit to project from the distal end opening, the projections at the at least two positions spaced from each other in the direction along the axis of the insertion hole always face an outer circumferential surface of the rigid shaft.

3. The medical manipulator system according to claim 2, wherein the projections are disposed at a first position and a second position that spaced from the first position and that located on a proximal end side relative to the first position, the first position being a position of a distal end of the rigid shaft in the longitudinal direction when a distal end of the treatment unit is in alignment with the distal end opening, or a position on the proximal end side of said position.

4. The medical manipulator system according to claim 3, wherein conditional formula (1) below is satisfied:
(D1−D2)/(2×L2)>(D2−d1)/L1 (1)
where
D1 represents an inner diameter of the rigid unit;
D2 represents an inner diameter of the two projections;
L1 represents a length from the second position to the first position;
L2 represents a maximum insertion amount of the rigid shaft from the first position; and
d1 represents an outer diameter of the rigid shaft.

5. The medical manipulator system according to claim 1, wherein the operation unit is equipped with:
a grip to be held by an operator, and
a force conversion mechanism configured to convert a rotation operation applied to the grip into movement of the treatment tool.

6. The medical manipulator system according to claim 5, wherein the force conversion mechanism and the rigid shaft are rotatably connected to each other about a longitudinal axis of the rigid shaft, and
the medical manipulator system further comprises a lock mechanism configured to lock rotation of the force conversion mechanism and the rigid shaft.

7. An access device configured to guide a treatment tool that includes a flexible shaft equipped with a treatment unit at a distal end thereof, and a rigid shaft connected to a proximal end of the flexible shaft, the access device comprising:
a flexible tube having an inner lumen into which the flexible shaft is to be inserted; and
a rigid unit connected to a proximal end of the flexible tube,
wherein:
the rigid unit includes an insertion hole into which the rigid shaft is to be inserted and which is connected to the inner lumen,
projections each projecting radially inward and having a particular length in a longitudinal axis direction are provided on an inner surface of the insertion hole, and
the projections are respectively provided at at least two positions spaced from each other in a direction along an axis of the insertion hole.

8. The access device according to claim 7,
wherein:
the flexible tube has a distal end opening from which the treatment unit projects; and
when the rigid shaft and the rigid unit are in such a positional relationship that allows the treatment unit to project from the distal end opening, the projections at the at least two positions spaced from each other in the direction along the axis of the insertion hole always face an outer circumferential surface of the rigid shaft.

9. The access device according to claim 8, wherein the projections are disposed at a first position and a second position spaced from the first position and located on a proximal end side relative to the first position, the first position being a position of a distal end of the rigid shaft in the longitudinal direction when a distal end of the treatment unit is in alignment with the distal end opening, or a position on the proximal end side relative to said position.

10. The access device according to claim 9, wherein conditional formula (1) below is satisfied:
(D1−D2)/(2×L2)>(D2−d1)/L1 (1)
where
D1 represents an inner diameter of the rigid unit;
D2 represents an inner diameter of the two projections;
L1 represents a length from the second position to the first position;
L2 represents a maximum insertion amount of the rigid shaft from the first position; and
d1 represents an outer diameter of the rigid shaft.

11. A medical manipulator system comprising:
a treatment tool; and
an access device configured to guide the treatment tool,
wherein the treatment tool includes
a flexible shaft equipped with a treatment unit at a distal end thereof,
a rigid shaft connected to a proximal end of the flexible shaft, and
an operation unit connected to a proximal end of the rigid shaft and configured to operate the treatment unit;
the access device includes
a flexible tube having an inner lumen into which the flexible shaft is to be inserted, and
a rigid unit connected to a proximal end of the flexible tube; and
the rigid unit includes
an insertion hole into which the rigid shaft is to be inserted and which is connected to the inner lumen;
an inner diameter of the insertion hole on a proximal end side in the longitudinal direction is smaller than that on a distal end side in the longitudinal direction.

12. The medical manipulator system according to claim 11, wherein:
a projection projecting radially inward and having a particular length in a longitudinal axis direction is formed on an inner surface of the insertion hole, the projection being disposed on a proximal end side with respect to a center position in the longitudinal direction, and
an inner diameter of the insertion hole at a position where the projection is located is smaller than that on a distal end side in the longitudinal direction.

13. The medical manipulator system according to claim 12, wherein the projection has a length that spans from a first position to a second position spaced from the first position and located on a proximal end side relative to the first position, the first position being a position of a distal end of the rigid shaft in the longitudinal direction when a distal end of the treatment tool is in alignment with a distal end opening of the flexible tube, or a position on the proximal end side relative to said position.

14. The medical manipulator system according to claim 13, wherein conditional formula (1) below is satisfied:
(D1−D2)/(2×L2)>(D2−d1)/L1 (1)
where
D1 represents an inner diameter of the rigid unit;
D2 represents an inner diameter of the projection;
L1 represents a length from the second position to the first position;
L2 represents a maximum insertion amount of the rigid shaft from the first position; and
d1 represents an outer diameter of the rigid shaft.

15. The medical manipulator system according to claim 11, wherein the operation unit is equipped with:
a grip to be held by an operator, and
a force conversion mechanism configured to convert a rotation operation applied to the grip into movement of the treatment tool.

16. The medical manipulator system according to claim 15, wherein the force conversion mechanism and the rigid shaft are rotatably connected to each other about a longitudinal axis of the rigid shaft, and the medical manipulator system further comprises a lock mechanism configured to lock rotation of the force conversion mechanism and the rigid shaft.
